(19) United States
(12) Patent Application Publication
(10) Pub. No.: US 2025/0176947 A1
(43) Pub. Date: Jun. 5, 2025

(54) APPARATUS, SYSTEM AND METHOD TO CONTROL AN ULTRASONIC IMAGING DEVICE BASED ON DYNAMIC PARAMETERS THEREOF

(71) Applicant: Exo Imaging, Inc., Santa Clara, CA (US)

(72) Inventors: Harishankar Prabhakaran, Redwood City, CA (US); Gordon T. Sasamori, Mountain View, CA (US)

(21) Appl. No.: 18/525,507

(22) Filed: Nov. 30, 2023

(51) Int. Cl. A61B 8/00; A61B 8/08

(52) U.S. Cl. CPC A61B 8/546; A61B 8/52; A61B 8/565

(57) ABSTRACT

An ultrasonic imaging probe, a computing device, methods, and computer-implemented media. The probe includes a plurality of ultrasonic transducer elements to generate and transmit ultrasonic waveforms in a direction of a target to be imaged; control circuitry to control the transducer elements; and communication circuitry to: send, for transmission from the probe to a computing device, an ultrasonic imaging probe signal including an identification (ID) of the probe, and one or more dynamic parameters of the probe; receive from the computing device a control signal based on the ultrasonic imaging probe signal. The probe further includes processing circuitry to cause an operational change at the probe based on the control signal, the operational change to cause at least one of a change in a power consumption by the probe, a change in a temperature at the probe, or a change in a battery status at the probe.

### FIELD

Embodiments relate in general to the field of ultrasonic imaging devices.

### BACKGROUND

Ultrasound imaging is widely used in the fields of medicine and non-destructive testing and may have a diagnostic or a procedural purpose. An ultrasonic imaging device, such as an ultrasonic imaging probe, may operate based on certain settings, such as various presets, imaging modes, frequencies, gains and/or associated imaging frame rates. The versatility of an ultrasonic imaging device as made possible in part by advances with respect to its settings has made the need for thermal management of such devices even more significant.

### SUMMARY

An ultrasonic imaging device of some embodiments may send an ultrasonic imaging device signal to a computing system to which it is communicatively paired. The ultrasonic imaging device signal may include identifying information to identify the device, and one or more dynamic parameters of the device, such as temperature or power consumption. The computing system may send a response signal to the device that is based on the ultrasonic imaging device signal. The response signal may include information to cause the device to implement operational changes that would result in a decrease in a temperature and/or a power consumption of the device.

### BRIEF DESCRIPTION OF THE DRAWINGS

The novel features of embodiments are set forth with particularity in the appended claims. A better understanding of the features and advantages of Some embodiments will be obtained by reference to the following detailed description that sets forth illustrative embodiments, in which the principles of the invention are utilized, and the accompanying drawings (also “Figure” and “FIG.” herein), of which:

FIG. 1 is a block diagram of an ultrasound imaging device in accordance with some embodiments.

FIG. 2 is a diagram of an ultrasound imaging system in accordance with some embodiments.

FIG. 3 is a schematic diagram of an ultrasound imaging device in accordance with some embodiments.

FIG. 4 depicts a flowchart of a process according to a first embodiment.

FIG. 5 depicts a flowchart of a process according to a second embodiment.

### DETAILED DESCRIPTION

Some embodiments advantageously provide an ultrasound imaging device, such as an ultrasonic imaging probe, that comprises: a plurality of ultrasonic transducer elements to generate and transmit ultrasonic waveforms in a direction of a target to be imaged; control circuitry to control the transducer elements; communication circuitry to: send, for transmission from the probe to a computing device, an ultrasonic imaging probe signal including an identification (ID) of the probe, and one or more dynamic parameters of the probe; receive from the computing device a control signal based on the ultrasonic imaging probe signal; and processing circuitry to cause an operational change at the probe based on the control signal, the operational change to cause at least one of a change in a power consumption by the probe, a change in a temperature at the probe, or a change in a battery status at the probe.

The sensor circuitry may send information based on the sensed dynamic parameters to a computing device that is paired with (e.g., in communication with), the probe, and the computing device is to send a response signal including a control signal to control operational parameters of the probe, for example to ensure a longer life for the probe.

Ultrasound imaging devices, such as handheld ultrasound imaging probes are used to perform an ultrasound imaging procedure on a patient. Typically, one hand is used to guide the ultrasound imaging device during scanning, another hand is used to interact with the computing user interface, such as a computing system that includes an ultrasound display, and a third hand may be required to control a medical tool such as a needle or catheter on a patient during a procedural ultrasound.

Some embodiments advantageously allow changing operational parameters of the probe using signals from a computing device that is paired with the probe. The computing device may receive one or more dynamic parameters from the Ultrasound imaging devices may be used to image internal tissue, bones, blood flow, or organs of human or animal bodies in a non-invasive manner. The images can then be displayed. To perform ultrasound imaging, the ultrasound imaging devices transmit an ultrasonic signal into the body and receive a reflected signal from the body part being imaged. Such ultrasound imaging devices include transducers and associated electronics, which may be referred to as transceivers or imagers, and which may be based on photo-acoustic or ultrasonic effects. Such transducers may be used for imaging and may be used in other applications as well. For example, the transducers may be used in medical imaging; flow measurements in arteries and pipes, can form speakers and microphone arrays; can perform lithotripsy; localized tissue heating for therapeutic; and highly intensive focused ultrasound (HIFU) surgery.

Additional aspects and advantages of some embodiments will become readily apparent to those skilled in this art from the instant detailed description, wherein only illustrative embodiments are shown and described. As will be realized, some embodiments are capable of achieving other, different goals, and their several details are capable of modifications in various obvious respects, all without departing from the disclosure. Accordingly, the drawings and description are to be regarded as illustrative in nature, and not as restrictive.

Traditionally, imaging devices such as ultrasound imagers used in medical imaging use piezoelectric (PZT) materials or other piezo ceramic and polymer composites. Such imaging devices may include a housing to house the transducers with the PZT material, as well as other electronics that form and display the image on a display unit. To fabricate the bulk PZT elements or the transducers, a thick piezoelectric material slab may be cut into large rectangular shaped PZT elements. These rectangular-shaped PZT elements may be expensive to build, since the manufacturing process involves precisely cutting generally the rectangular-shaped thick PZT or ceramic material and mounting it on substrates with precise spacing. Further, the impedance of the transducers is much higher than the impedance of the body tissue, which can affect performance.

Still further, such thick bulk PZT elements can require very high voltage pulses, for example 100 volts (V) or more to generate transmission signals. This high drive voltage can sometimes result in high power dissipation, since the power dissipation in the transducers is proportional to the square of the drive voltage. This high power dissipation generates heat within the ultrasound imaging device such that cooling arrangements are necessitated. These cooling systems increase the manufacturing costs and weights of the ultrasound imaging devices which makes the ultrasound imaging devices more burdensome to operate.

Some embodiments may be utilized in the context of imaging devices that utilize either piezoelectric micromachined ultrasound transducer (pMUT) or capacitive micromachined ultrasonic transducer (cMUT) technologies, as described in further detail herein.

In general, MUTs, such as both cMUTs and pMUTs, include a diaphragm (a thin membrane attached at its edges, or at some point in the interior of the probe), whereas a “traditional,” bulk PZT element typically consists of a solid piece of material.

Piezoelectric micromachined ultrasound transducers (pMUTs) may be efficiently formed on a substrate leveraging various semiconductor wafer manufacturing operations. Semiconductor wafers may come in 6 inch, 8 inch, and 12 inch sizes and are capable of housing hundreds of transducer arrays. These semiconductor wafers start as a silicon substrate on which various processing operations are performed. An example of such an operation is the formation of SiO2 layers, also known as insulating oxides. Various other operations such as the addition of metal layers to serve as interconnects and bond pads are performed to allow connection to other electronics. Yet another example of a machine operation is the etching of cavities. Compared to the conventional transducers having bulky piezoelectric material, pMUT elements built on semiconductor substrates are less bulky, are cheaper to manufacture, and have simpler and higher performance interconnection between electronics and transducers. As such, they provide greater flexibility in the operational frequency of the ultrasound imaging device using the same, and potential to generate higher quality images. Frequency response may for example be expanded though flexibility of shaping the diaphragm and its active areas with piezo material.

In some embodiments, the ultrasound imaging device includes an application specific integrated circuit (ASIC) that includes transmit drivers, sensor circuitry for received echo signals, and control circuitry to control various operations. The ASIC may be formed on the same or another semiconductor wafer. This ASIC may be placed in close proximity to pMUT or cMUT elements to reduce parasitic losses. As a specific example, the ASIC may be 50 micrometers (μm) or less away from the transducer array. In a broader example, there may be less than 100 μm separation between the 2 wafers or 2 die, where each wafer includes many die, and a die includes a transducer array in the transducer wafer and an ASIC array in the ASIC wafer. The array may have up to 10,000 or more individual elements. In some embodiments, the ASIC has matching dimensions relative to the pMUT or cMUT array and allows the devices to be stacked for wafer-to-wafer interconnection or transducer die on ASIC wafer or transducer die to ASIC die interconnection. Alternatively, the transducer can also be developed on top of the ASIC wafer using low temperature piezo material sputtering and other low temperature processing compatible with ASIC processing.

Wherever the ASIC and the transducer interconnect, according to one embodiment, the two may have similar footprints. More specifically, according to the latter embodiment, a footprint of the ASIC may be an integer multiple or divisor of the MUT footprint.

Regardless of whether the ultrasound imaging device is based on pMUT or cMUT, an imaging device according to some embodiments may include a number of transmit channels and a number of receive channels. Transmit channels are to drive the transducer elements with a voltage pulse at a frequencies the elements are responsive to. This may cause an ultrasonic waveform to be emitted from the elements, which waveform is to be directed towards an object to be imaged (target object), such as toward an organ or other tissue in a body. In some examples, the ultrasound imaging device with the array of transducer elements may make mechanical contact with the body using a gel in between the ultrasound imaging device and the body. The ultrasonic waveform travels towards the object or target, i.e., an organ, and a portion of the waveform is reflected back to the transducer elements in the form of received/reflected ultrasonic energy where the received ultrasonic energy may be converted to an electrical energy within the ultrasound imaging device. The received ultrasonic energy may be processed by a number of receive channels to convert the received ultrasonic energy to signals, and the signals may be processed by other circuitry to develop an image of the object for display based on the signals.

An embodiment of an ultrasound imaging device includes a transducer array, and control circuitry including, for example, an application-specific integrated circuit (ASIC), and transmit and receive beamforming circuitry, and optionally additional control electronics.

In an embodiment, an imaging device may include a handheld casing or handheld housing where transducers and associated electronic circuitries, such as a control circuitry and optionally a computing device are housed. The ultrasound imaging device may also contain a battery to power the electronic circuitries.

Thus, some embodiments pertain to a portable imaging device utilizing either pMUT elements or cMUT elements in a 2D array. In some embodiments, such an array of transducer elements is coupled to an application specific integrated circuit (ASIC) of the ultrasound imaging device.

An ultrasound exam (or “scanning operation”) may be associated with one of a number of ultrasound presets. An individual ultrasound preset may be associated with one or more ultrasound settings. Each setting can be changed or controlled independently of other settings within a preset to improve ultrasonic imaging.

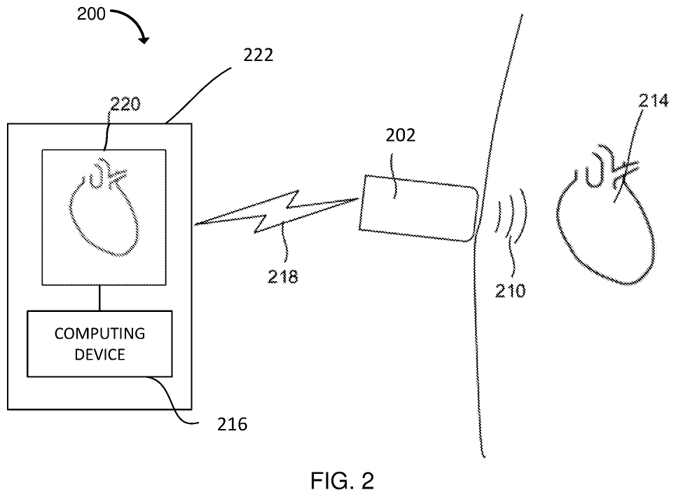

Some examples of ultrasound settings include:
a) Gain: the gain setting may control the brightness of the ultrasonic image shown on a display, such as display 220 of FIG. 2. Increasing the gain can make the image brighter, while decreasing the gain can make the image darker.
b) Depth: the depth setting controls how deep the ultrasound waves penetrate into the body. Increasing the depth can allow for imaging deeper structures, while decreasing the depth can provide more detailed imaging of shallow structures.
c) Frequency: the frequency setting controls the frequency of the ultrasound waves per transducer element. Higher frequency ultrasound waves provide better image resolution for superficial structures, while lower frequency ultrasound waves can penetrate deeper into the body for imaging deeper structures.
d) Time gain compensation (TGC): TGC settings adjust the gain of the ultrasound signal at different depths. This helps to ensure that the image is uniformly bright at all depths.
e) Dynamic range: the dynamic range setting adjusts the range of brightness levels that can be displayed in the image. Increasing the dynamic range can improve the visibility of subtle differences in tissue texture or echogenicity.
f) Focus: the focus setting adjusts where the ultrasound waves converge in the body. This can help to improve image resolution and clarity at a specific depth.
g) Harmonics: the harmonics setting allows the probe to emit and receive sound waves at higher frequencies than a fundamental frequency of the probe. Harmonics are multiples of the fundamental frequency, and they can be used to provide higher resolution images in certain medical imaging applications. In medical ultrasonography, the fundamental frequency of a probe may be in the range between about 2 to about 10 MHz.
h) Mode:
   1. Doppler: the Doppler mode of an ultrasonic imaging probe refers to a mode that allows the probe to detect and measure the velocity and direction of blood flow in the body. This is done by using the Doppler effect, which is the change in frequency of sound waves that occurs when they are reflected off moving objects. In medical ultrasonography, the Doppler setting is often used to assess blood flow in arteries and veins. The probe emits sound waves at a specific frequency that is reflected off the moving red blood cells in the vessel. The returning sound waves are then analyzed to determine the velocity and direction of the blood flow.
   2. B-mode: the B mode produces a two-dimensional grayscale image of the body. B-mode may use a single transducer to emit sound waves and then receive the reflected echoes to create an image.
   3. M-mode: This mode is used to visualize motion within a specific region of the body over time. It produces a one-dimensional image in which the x-axis represents time and the y-axis represents the position of the moving structure.
   4. 3D/4D mode: This mode is used to create three-dimensional images of the body, allowing for better visualization and assessment of complex anatomical structures. 4D mode adds the element of time to the 3D image, producing a real-time view of the body in motion.
i) Focal zone: Focal zone pertains to the region in the body where the ultrasonic beam is focused to achieve the best image quality. The focal zone is determined by a transducer element's aperture and the frequency of the emitted sound waves. Typically, an ultrasonic imaging probe has multiple focal zones that can be adjusted by the user.
j) Persistence: Persistence is a feature that enables the display of previous ultrasound frames on the screen in a semi-transparent manner. This can help reduce speckle noise and enhance the visualization of structures that are moving slowly.
k) Automatic gain control (AGC): This is a feature that adjusts the gain of the ultrasound signal to maintain a consistent brightness level across the entire image. AGC can be set to “auto” or “manual” mode, and some probes have additional features such as tissue equalization, which can further enhance image quality.
l) Spatial compounding: Spatial compounding is a technique that involves combining multiple ultrasound images from different angles to create a single composite image with reduced noise and improved contrast resolution.
m) Frequency compounding: Frequency compounding is a technique that involves using different frequencies of ultrasound waves to create a single image. This can improve image quality by reducing the effects of attenuation and scattering of sound waves in tissues.
n) Sine functions: Sine functions are used in ultrasonic imaging to modulate the amplitude, frequency, or phase of the emitted sound waves. This can help to reduce artifacts and improve the quality of the resulting image.
o) Line density: Line density refers to the number of scan lines used to create an image. Higher line density can improve image resolution but also increases the amount of data that needs to be processed.
p) Tint maps or tint maps configuration: Tint maps are color-coded maps used to display different types of information in an ultrasound image. For example, different shades of blue and red can be used to indicate the direction and velocity of blood flow in a vessel. Tint maps can help to make it easier for the user to interpret the information presented in the image.

Other examples of settings include: measurements, annotations, settings for tissue border delineation, to name a few.

A given preset may be associated with a cluster of ultrasound settings. Examples of presets for ultrasonic imaging include:
i. Abdominal: This preset may be optimized for imaging the abdominal region, including the liver, pancreas, and kidneys. It typically uses a low to medium frequency and a wide dynamic range to capture details in the deep structures of the abdomen.
ii. Renal: This preset may be specifically optimized for imaging the kidneys and detecting any abnormalities such as cysts, tumors, or obstructions. It typically uses a medium frequency and has specialized settings for measuring renal blood flow and identifying renal stones.
iii. Obstetrics: This preset may be optimized for imaging the fetus during pregnancy. It typically uses a high frequency and has specialized settings for measuring fetal biometry and assessing fetal growth and development.
iv. Cardiac: This preset may be optimized for imaging the heart and cardiovascular system. It typically uses a high frequency and specialized settings for measuring blood flow, cardiac function, and detecting abnormalities such as valve stenosis or regurgitation.
V. Musculoskeletal: This preset may be optimized for imaging the musculoskeletal system, including bones, joints, and soft tissue. It typically uses a high frequency and has specialized settings for detecting fractures, tendon tears, and other soft tissue injuries.
vi. Breast: This preset may be optimized for imaging the breast and detecting abnormalities such as tumors or cysts. It typically uses a high frequency and has specialized settings for detecting microcalcifications and characterizing breast lesions.
vii. Thyroid: This preset may be optimized for imaging the thyroid gland and detecting abnormalities such as nodules or cysts. It typically uses a high frequency and has specialized settings for measuring thyroid volume and blood flow.

The above are just a few examples of presets for medical ultrasonic imaging probes. Different manufacturers and models of probes may have different presets or customizable settings based on the specific imaging needs of the user.

In the following description, for purposes of explanation, specific details are set forth in order to provide an understanding of the disclosure. It will be apparent, however, to one skilled in the art that the disclosure may be practiced without these details. Furthermore, one skilled in the art will recognize that examples of the present disclosure, described below, may be implemented in a variety of ways, such as a process, one or more processors (processing circuitry) of a control circuitry, one or more processors (or processing circuitry) of a computing device, a system, a device, or a method on a tangible computer-readable medium.

One skilled in the art shall recognize: (1) that certain fabrication operations may optionally be performed; (2) that operations may not be limited to the specific order set forth herein; and (3) that certain operations may be performed in different orders, including being done contemporaneously, and (4) operations may involve using Artificial Intelligence.

Elements/components shown in diagrams are illustrative of exemplary embodiments and are meant to avoid obscuring the disclosure. Reference in the specification to “one example,” “preferred example,” “an example,” “examples,” “an embodiment,” “some embodiments,” or “embodiments” means that a particular feature, structure, characteristic, or function described in connection with the example is included in at least one example of the disclosure and may be in more than one example. The appearances of the phrases “in one example,” “in an example,” “in examples,” “in an embodiment,” “in some embodiments,” or “in embodiments” in various places in the specification are not necessarily all referring to the same example or examples. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims. Furthermore, the use of certain terms in various places in the specification is for illustration and should not be construed as limiting.

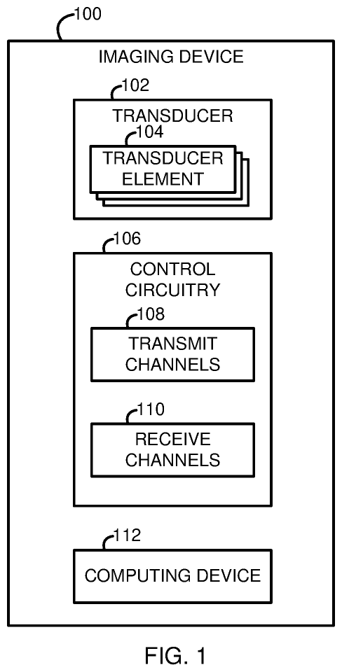

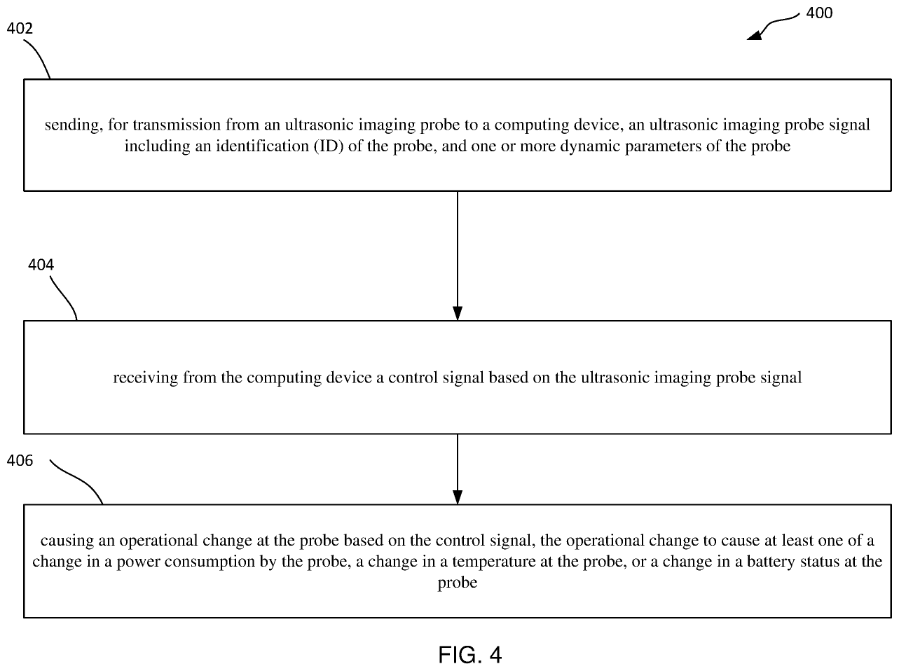

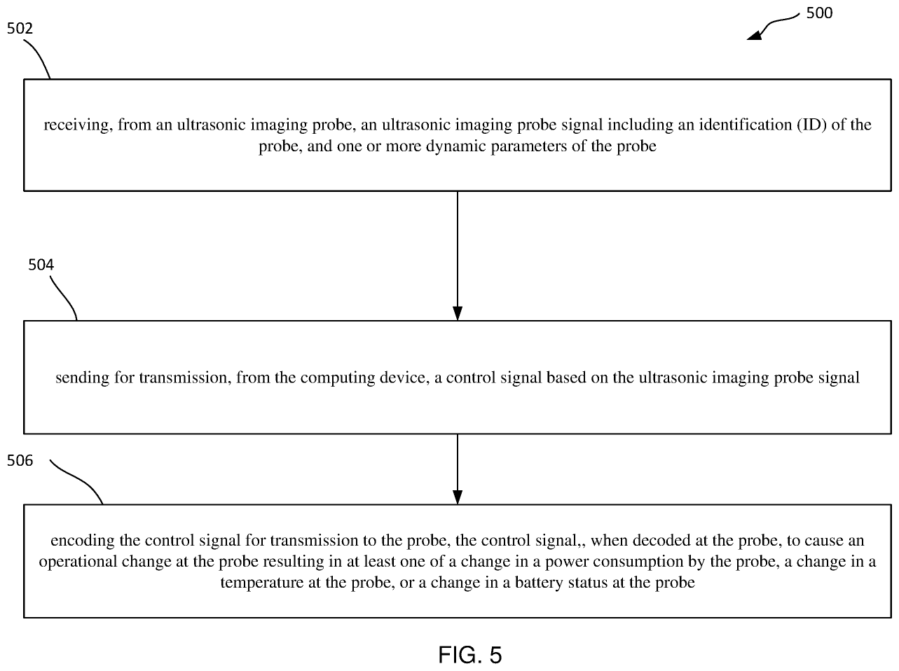

Reference will be made to FIGS. 1-3, which show devices and circuitries that may be used to implement some embodiments as described herein. Reference will further be made to FIGS. 4A and 4B which show an ultrasound imaging device according to the state of the art. Reference will thereafter be made to FIGS. 5A and 5B, which show an ultrasound imaging device according to one embodiment being held in two different manners.

Turning now to the figures, FIG. 1 is a block diagram of an imaging device 100 with a controller or control circuitry 106 controlling selectively alterable channels (108, 110) and having imaging computations performed on a computing device 112 according to principles described herein. As described above, the ultrasound imaging device 100 may be used to generate an image of internal tissue, bones, blood flow, or organs of human or animal bodies. Accordingly, the ultrasound imaging device 100 may transmit a signal into the body and receive a reflected signal from the body part being imaged. Such imaging devices may include either pMUT or cMUT, which may be referred to as transducers or imagers, which may be based on photo-acoustic or ultrasonic effects. The ultrasound imaging device 100 may be used to image other objects as well. For example, the ultrasound imaging device may be used in medical imaging; flow measurements in pipes, speaker, and microphone arrays; lithotripsy; localized tissue heating for therapeutic; and highly intensive focused ultrasound (HIFU) surgery.

In addition to use with human patients, the ultrasound imaging device 100 may be used to acquire an image of internal organs of an animal as well. Moreover, in addition to imaging internal organs, the ultrasound imaging device 100 may also be used to determine direction and velocity of blood flow in arteries and veins as in Doppler mode imaging and may also be used to measure tissue stiffness.

The ultrasound imaging device 100 may be used to perform different types of imaging. For example, the ultrasound imaging device 100 may be used to perform one-dimensional imaging, also known as A-Scan, two-dimensional imaging, also known as B scan, three-dimensional imaging, also known as C scan, and Doppler imaging (that is, the use of Doppler ultrasound to determine movement, such as fluid flow within a vessel). The ultrasound imaging device 100 may be switched to different imaging modes, including without limitation linear mode and sector mode, and electronically configured under program control.

To facilitate such imaging, the ultrasound imaging device 100 includes one or more ultrasound transducers 102, each transducer 102 including an array of ultrasound transducer elements 104. Each ultrasound transducer element 104 may be embodied as any suitable transducer element, such as a pMUT or cMUT element. The transducer elements 104 operate to 1) generate the ultrasonic pressure waves that are to pass through the body or other mass and 2) receive reflected waves (received ultrasonic energy) off the object within the body, or other mass, to be imaged. In some examples, the ultrasound imaging device 100 may be configured to simultaneously transmit and receive ultrasonic waveforms or ultrasonic pressure waves (pressure waves in short). For example, control circuitry 106 may be configured to control certain transducer elements 104 to send pressure waves toward the target object being imaged while other transducer elements 104, at the same time, receive the pressure waves/ultrasonic energy reflected from the target object, and generate electrical charges based on the same in response to the received waves/received ultrasonic energy/received energy.

In some examples, each transducer element 104 may be configured to transmit or receive signals at a certain frequency and bandwidth associated with a center frequency, as well as, optionally, at additional center frequencies and bandwidths. Such multi-frequency transducer elements 104 may be referred to as multi-modal elements 104 and can expand the bandwidth of the ultrasound imaging device 100. The transducer element 104 may be able to emit or receive signals at any suitable center frequency, such as about 0.1 to about 100 megahertz.

To generate the pressure waves, the ultrasound imaging device 100 may include a number of transmit (Tx) channels 108 and a number of receive (Rx) channels 110. The transmit channels 108 may include a number of components that drive the transducer 102, i.e., the array of transducer elements 104, with a voltage pulse at a frequency that they are responsive to. This may cause an ultrasonic waveform to be emitted from the transducer elements 104 towards an object to be imaged.

According to some embodiments, an ultrasonic waveform may include one or more ultrasonic pressure waves transmitted from one or more corresponding transducer elements of the ultrasound imaging device substantially simultaneously.

The ultrasonic waveform travels towards the object to be imaged (target) and a portion of the waveform is reflected back to the transducer 102, which converts it to an electrical energy through a piezoelectric effect. The receive channels 110 collect electrical energy thus obtained, and process it, and send it for example to the computing device 112, which develops or generates an image that may be displayed.

In some examples, the number of transmit channels 108 and receive channels 110 in the ultrasound imaging device 100 may remain constant, although the coupling of respective transducer elements to the transmit channels 108 and receive channels 110 may vary, for example based on coupling schemes dictated by the control circuitry. A coupling of the transmit and receive channels to the transducer elements may be, in one embodiment, controlled by control circuitry 106. In some examples, for example as shown in FIG. 1, the control circuitry may include the transmit channels 108 and the receive channels 110. For example, the transducer elements 104 of a transducer 102 may be formed into a two-dimensional spatial array with N columns and M rows. In a specific example, the two-dimensional array of transducer elements 104 may have 128 columns and 32 rows. In this example, the ultrasound imaging device 100 may have up to 128 transmit channels 108 and up to 128 receive channels 110. In this example, each transmit channel 108 and receive channel 110 may be coupled to multiple or single pixels 104. For example, depending on the imaging mode (for example, whether a linear mode where a number of transducers transmit ultrasound waves in a same spatial direction, or a sector mode, where a number of transducers transmit ultrasound waves in different spatial directions), each column of transducer elements 104 may be coupled to a single transmit channel 108 and a single receive channel (110). In this example, the transmit channel 108 and receive channel 110 may receive composite signals, which composite signals combine signals received at each transducer element 104 within the respective column. In another example, i.e., during a different imaging mode, each transducer element 104 may be coupled to its dedicated transmit channel 108 and its dedicated receive channel 110. In some embodiments, a transducer element 104 may be coupled to both a transmit channel 108 and a receive channel 110. For example, a transducer element 104 may be adapted to create and transmit an ultrasound pulse and then detect the echo of that pulse in the form of converting the reflected ultrasonic energy into electrical energy.

The control circuitry 106 may be embodied as any circuit or circuits configured to perform the functions described herein. For example, the control circuitry 106 may be embodied as or otherwise include an application specific integrated circuit (ASIC), a field programmable gate array (FPGA), a system-on-a-chip, a processor and memory, a voltage source, a current source, one or more amplifiers, one or more digital-to-analog converters, one or more analog-to-digital converters, etc.

The illustrative computing device 112 may be embodied as any suitable computing device including any suitable components, such as one or more processors (i.e. one or more processing circuitries), one or more memory circuitries, one or more communication circuitries, one or more batteries, one or more displays, etc. In one embodiment, the computing device 112 may be integrated with the control circuitry 106, transducers 102, etc., into a single microelectronic package or single chip, or a single system on a chip (SoC), or a single ultrasound imaging device housing as suggested for example in the embodiment of FIG. 1. In other embodiments, some or all of the computing devices may be in a separate microelectronic package from the control circuitry, or in a separate device distinct from the ultrasound imaging device such as an ultrasound imaging probe, as suggested for example in the embodiment of in FIG. 2 as will be described in further detail below.

Each transducer element may have any suitable shape such as, square, rectangle, ellipse, or circle. The transducer elements may be arranged in a two dimensional array arranged in orthogonal directions, such as in N columns and M rows as noted herein or may be arranged in an asymmetric (or staggered) rectilinear array.

Transducer elements 104 may have associated transmit driver circuits of associated transmit channels, and low noise amplifiers of associated receive channels. Thus, a transmit channel may include transmit drivers, and a receive channel may include one or more low noise amplifiers. For example, although not explicitly shown, the transmit and receive channels may each include multiplexing and address control circuitry to enable specific transducer elements and sets of transducer elements to be activated, deactivated or put in low power mode. It is understood that transducers may be arranged in patterns other than orthogonal rows and columns, such as in a circular fashion, or in other patterns based on the ranges of ultrasonic waveforms to be generated therefrom.

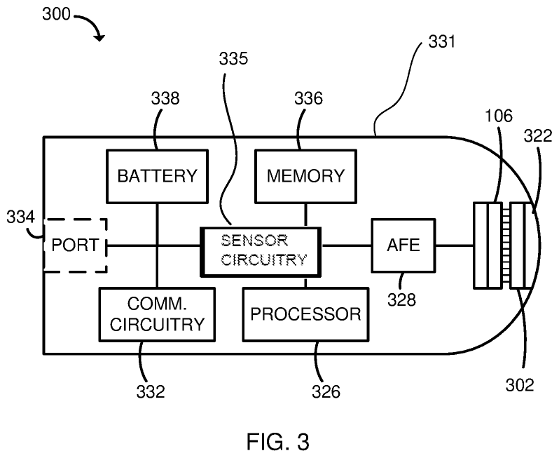

FIG. 2 is a diagram of an imaging environment including an imaging system 200 with selectively configurable characteristics, according to an embodiment. The imaging system of FIG. 2 may include an ultrasound imaging device 202 (which may be similar to ultrasonic imaging probe 300 described below in the context of FIG. 3) and a computing system 222 which includes a computing device 216 and a display 220 coupled to the computing device, as will be described in further detail below.

As depicted in FIG. 2, the computing device 216 may, according to one embodiment, and unlike the embodiment of FIG. 1, be physically separate from the ultrasound imaging device 220. For example, the computing device 216 and display device 220 may be disposed within a separate device (in this context, the shown computing system 222, physically separate from imaging device 202 during operation) as compared with the components of the ultrasound imaging device 202. The computing system 222 may include a mobile device, such as cell phone or tablet, or a stationary computing device, which can display images to a user. In another example, as shown in FIG. 1 for example, the display device, the computing device, and associated display, may be part of the ultrasound imaging device 202 (now shown). That is, the ultrasound imaging device 100, computing device 216, and display device 220 may be disposed within a single housing.

In one example, the computing system, such as computing system 222 of FIG. 2, may include a host processor device coupled to the computing device; a display communicatively coupled to a host processor; a network interface communicatively coupled to the host processor; or a battery to power the system.

A “computing device” as referred to herein may, in some embodiments, be configured to generate signals to at least one of cause an image of the object to be displayed on a display, or cause information regarding the image to be communicated to a user.

A “computing device,” as referred to herein may, in some embodiments, be configured to receive, from an ultrasonic imaging probe, signals carrying information regarding the ultrasonic imaging probe (hereinafter “ultrasonic imaging probe parameter signals”), and to process those ultrasonic imaging probe parameter signals to cause generation of control signals to change parameters of the ultrasonic imaging probe.

A “parameter of an ultrasonic imaging probe” or “ultrasonic imaging probe parameter” as used herein refers to information regarding the ultrasonic imaging probe as a device. An ultrasonic imaging probe parameter may include a static parameter, that is, information regarding the ultrasonic imaging probe that is fixed, such as its identification (ID), serial number, place of manufacture, date of manufacture, processor IDs, etc. An ultrasonic imaging probe parameter may include a dynamic parameter, that is, information about the ultrasonic imaging probe that is subject to change. Examples of dynamic parameters include, for example, temperature, voltage, power, whether or not paired to a computing device, location (including geographic and/or indoor location and movement such as speed and direction), battery charge percentage/remaining battery life.

As depicted, the imaging system includes the ultrasound imaging device 202 that is configured to generate and transmit, via the transmit channels (FIG. 1, 108), pressure waves 210 toward an object, such as a heart 214, in a transmit mode/process. The internal organ, or other object to be imaged, may reflect a portion of the pressure waves 210 toward the ultrasound imaging device 202 which may receive, via a transducer (such as transducer 102 of FIG. 1), receive channels (FIG. 1, 110), control circuitry (FIG. 1, 106), the reflected pressure waves. The transducer may generate an electrical signal based on the received ultrasonic energy in a receive mode/process. A transmit mode or receive mode may be applicable in the context of imaging devices that may be configured to either transmit or receive, but at different times. However, as noted previously, some imaging devices according to embodiments may be adapted to be in both a transmit mode and a receive mode simultaneously. The system also includes a computing device 216 that is to communicate with the ultrasound imaging device 100 through a communication channel, such as a wireless communication channel 218 as shown, although embodiments also encompass within their scope wired communication between a computing system and imaging device. The ultrasound imaging device 100 may communicate signals to the computing device 216 which may have one or more processors to process the received signals to complete formation of an image of the object. A display device 220 of the computing system 222 may then display images of the object using the signals from the computing device.

An imaging device according to some embodiments may include a portable device, and/or a handheld device that is adapted to communicate signals through a communication channel, either wirelessly (using a wireless communication protocol, such as an IEEE 802.11 or Wi-Fi protocol, a Bluetooth protocol, including Bluetooth Low Energy, a mmWave communication protocol, or any other wireless communication protocol as would be within the knowledge of a skilled person) or via a wired connection such as a cable (such as USB2, USB 3, USB 3.1, and USB-C) or such as interconnects on a microelectronic device, with the computing device. In the case of a tethered or wired, connection, the ultrasound imaging device may include a port for receiving a cable connection of a cable that is to communicate with the computing device. In the case of a wireless connection, the ultrasound imaging device 100 may include a wireless transceiver to communicate with the computing device 216.

It should be appreciated that, in various embodiments, different aspects of the disclosure may be performed in different components. For example, in one embodiment, the ultrasound imaging device may include circuitry (such as the channels) to cause ultrasound waveforms to be sent and received through its transducers, while the computing device may be adapted to control such circuitry to the generate ultrasound waveforms at the transducer elements of the ultrasound imaging device using voltage signals, and further a processing of the received ultrasonic energy.

FIG. 3 represents a view of an imaging device according to some embodiments, as will be described in further detail below.

As seen in FIG. 3, the ultrasonic imaging probe 300 may include a handheld casing or housing 331 where transducers 302 and associated electronics are housed. The ultrasound imaging device may also contain a battery 338 to power the electronics. FIG. 3 thus shows an embodiment of a portable imaging device capable of 2D and 3D imaging using pMUTs in a 2D array, optionally built on a silicon wafer. Such an array coupled to an application specific integrated circuit (ASIC) 106 with electronic configuration of certain parameters, enables a higher quality of image processing at a low cost than has been previously possible. Further by controlling certain parameters, for example the number of channels used, power consumption may be altered, and temperature may be changed.

FIG. 3 is a schematic diagram of an imaging device such as an ultrasonic imaging probe 300 with selectively adjustable features, according to some embodiments. The ultrasonic imaging probe 300 may be similar to imaging device 100 of FIG. 1, or to imaging device 202 of FIG. 2, by way of example only. FIG. 3 depicts transducer(s) 302 of the ultrasonic imaging probe 300. As described above, the transducer(s) 302 may include arrays of transducer elements (FIG. 1, 104) that are adapted to transmit and receive pressure waves (FIG. 2, 210). In some examples, the ultrasonic imaging probe 300 may include a coating layer 322 that serves as an impedance matching interface between the transducers 302 and the human body, or other mass or tissue through which the pressure waves (FIG. 2, 210) are transmitted. In some cases, the coating layer 322 may serve as a lens when designed with the curvature consistent with focal length desired.

The ultrasonic imaging probe 300 housing 331 may be embodied in any suitable form factor. In some embodiments, part of the ultrasonic imaging probe 300 that includes the transducers 302 may extend outward from the rest of the ultrasonic imaging probe 300. The ultrasonic imaging probe 300 may be embodied as any suitable ultrasonic medical probe, such as a convex array probe, a micro-convex array probe, a linear array probe, an endovaginal probe, endorectal probe, a surgical probe, an intraoperative probe, etc.

In some embodiments, the user may apply gel on the skin of a living body before a direct contact with the coating layer 322 so that the impedance matching at the interface between the coating layer 322 and the human body may be improved. Impedance matching reduces the loss of the pressure waves (FIG. 2, 210) at the interface and the loss of the reflected wave travelling toward the ultrasonic imaging probe 300 at the interface.

In some examples, the coating layer 322 may be a flat layer to maximize transmission of acoustic signals from the transducer(s) 102 to the body and vice versa. The thickness of the coating layer 322 may be a quarter wavelength of the pressure wave (FIG. 2, 210) to be generated at the transducer(s) 102.

The ultrasonic imaging probe 300 also includes a control circuitry 106, such as one or more processors, optionally in the form of an application-specific integrated circuit (ASIC chip or ASIC), for controlling the transducers 102. The control circuitry 106 may be coupled to the transducers 102, such as by way of bumps.

The ultrasonic imaging probe 300 includes sensor circuitry 335 coupled to the communication circuitry 332 and to the processor circuitry 326. The sensor circuitry 335 may include one or more sensor circuitries to sense one or more dynamic parameters of the ultrasonic imaging probe.

The ultrasonic imaging probe may also include one or more processors (or processing circuitries) 326 for controlling the components of the ultrasonic imaging probe 300. One or more processors 326 may be configured to, in addition to control circuitry 106, at least one of control an activation of transducer elements, process signals based on reflected ultrasonic waveforms from the transducer elements or generate signals to cause generation of an image of an object being imaged by one or more processors of a computing device, such as computing device 112 of FIG. 1 or 216 of FIG. 2. One or more processors 326 may further be adapted to perform other processing functions associated with the ultrasonic imaging probe.

The one or more processors 326 may be embodied as any type of processors 326. For example, the one or more processors 326 may be embodied as a single or multi-core processor(s), a single or multi-socket processor, a digital signal processor, a graphics processor, a neural network compute engine, an image processor, a microcontroller, a field programmable gate array (FPGA), or other processor or processing/controlling circuit.

The ultrasonic imaging probe 300 may also include circuitry 328, such as Analog Front End (AFE), for processing/conditioning signals. The analog front end 328 may be embodied as any circuit or circuits configured to interface with the control circuitry 106 and other components of the ultrasonic imaging probe, such as the processing circuitry 326. For example, the analog front end 328 may include, e.g., one or more digital-to-analog converters, one or more analog-to-digital converters, one or more amplifiers, etc.

The ultrasonic imaging probe may include a communication circuitry 332 for communicating data, including control signals, with an external device, such as the computing device (FIG. 2, 216), through for example an input/output (I/O) circuitry 334 of the probe 300. The communication circuitry 332 may, for example, include signal processing circuitry for signals communicated through the I/O circuitry. For example, the communication circuitry may include one or more of a baseband processor, a modem, digital signal processing (DSP) circuitry, an analog-to-digital converter (ADC), or an equalizer circuitry.

For example, the I/O circuitry 334 may include one or more ports for wired communication with the probe 300, or a wireless transceiver circuitry 335 for wireless communication with the probe 300. The wireless transceiver circuitry 335 may, for example, include one or more transmit (TX) circuitries to transmit signals from the ultrasonic imaging probe 300, and one or more receive (RX) circuitries to receive signals into the ultrasonic imaging probe 300. The TX and RX circuitries may include TX and RX ports within port 334, which may, for example, correspond to a USB port. The TX and RX circuitries may include TX chains and RX chains of one or more wireless transceivers. A TX chain or an RX chain may include, for example one or more antennas, amplifiers, filters, and/or mixers.

The ultrasonic imaging probe 300 may include memory circuitry 336 for storing data. The memory circuitry 336 may be embodied as any type of volatile or non-volatile memory or data storage capable of performing the functions described herein. In operation, the memory circuitry 336 may store various data and software used during operation of the ultrasonic imaging probe 300 such as operating systems, applications, programs, libraries, and drivers.

In some examples, the ultrasonic imaging probe 300 may include a battery 338 for providing electrical power to the components of the ultrasonic imaging probe 300. The battery 338 may also include battery charging circuits which may be wireless or wired charging circuits (not shown). The ultrasonic imaging probe may include a gauge that indicates a battery charge consumed and is used to configure the ultrasonic imaging probe to optimize power management for improved battery life. Additionally or alternatively, in some embodiments, the ultrasonic imaging probe may be powered by an external power source, such as by plugging the ultrasonic imaging probe into a wall outlet.

The ultrasonic imaging probe may further include sensor circuitry 335, which may be configured to sense one or more dynamic parameters of the ultrasonic imaging probe, such as temperature, voltage, electrical current, location (including movement), pairing information regarding pairing to one or more computing devices (such as computing device 216 of FIG. 2), number of scans performed (including number of scans of respective scan types performed). The sensor circuitry may include one or more circuitries. Where the sensor circuitry 335 includes a plurality of distinct circuitries, each such circuitry may be adapted to sense a different dynamic parameter of the ultrasonic imaging probe, and each such circuitry may be positioned in different location of the probe.

The sensor circuitry 335 may for example be configured to sense temperature at any of the components and/or subcomponents of the ultrasonic imaging probe 300.

For example, the sensor circuitry 335 may be configured to sense one or more temperatures of the transducer elements of transducer 302.

For example, the sensor circuitry 335 may be configured to sense one or more temperatures of the TX channels 108 and/or of the RX channels 110 (see FIG. 1) that drive the transducer elements of transducers 302.

For example, the sensor circuitry 335 may be configured to sense one or more temperatures of the control circuitry 106 that drives the transducer elements of transducers 302 by way of TX channels 108 and RX channels 110.

For example, the sensor circuitry may be configured to sense one or more temperatures of the I/O circuitry, such as one or more temperatures of the TX circuitry and/or the RX circuitry. For example, the sensor circuitry may be configured to sense one or more temperatures of a wireless transceiver circuitry, such as a temperature at the TX chains and/or a temperature at the RX chains of the wireless transceiver circuitry.

For example, the sensor circuitry may be configured to sense one or more temperatures at processing circuitry 326.

For example, the sensor circuitry may be configured to sense one or more temperatures at battery 338.

For example, the sensor circuitry may be configured to detect a temperature of the probe, and the processing circuitry may then cause the probe to send information regarding such temperature to the computing device.

For example, the sensor circuitry may be configured to detect a temperature of the probe, and the processing circuitry may then cause the probe to send information regarding such temperature to the memory circuitry 336, including information on not only the temperature, but also on at least one of the date, time, and/or duration of the temperature. Thus, memory circuitry 336 could store information on a history of temperatures of the probe 300. According to an embodiment, the probe 300 may send such information regarding the history of temperatures to a computing device to which it is paired, or to a cloud network. According to an embodiment, processing circuitry 326 of probe 300 may cause a set of temperatures stored in the memory circuitry 336 to be sent to the computing device, the set corresponding for example to temperatures corresponding to control circuitry 106, or to a group of transducer elements 302, or to one or more TX or RX circuitries of the I/O circuitry 334, or to a given preset or setting of the probe 300, by way of example.

The sensor circuitry may for example be configured to sense voltage at any of the components and/or subcomponents of the ultrasonic imaging probe 300.

For example, the sensor circuitry 335 may be configured to sense voltage across battery 338.

For example the sensor circuitry 335 may be configured to sense voltage at one or more components of control circuitry 106.

For example, the sensor circuitry may be configured to detect a voltage at the probe, and the processing circuitry may then cause the probe to send information regarding such voltage to the computing device.

For example, the sensor circuitry may be configured to detect a voltage at the probe, and the processing circuitry may then cause the probe to send information regarding such temperature to the memory circuitry 336.

The sensor circuitry may for example be configured to sense a percentage charge of battery 338, for example using voltage and/or current measurements across the battery 338.

For example, the sensor circuitry may be configured to detect a charge status of the battery, and the processing circuitry 326 may then cause the probe to send information regarding such charge status to the computing device.

For example, the sensor circuitry may be configured to detect a charge status of the battery, and the processing circuitry 326 may then cause the probe to send information regarding such charge status to the memory circuitry 336, including information on not only the charge status, but also on at least one of the date, time, and/or duration of the charge status. Thus, memory circuitry 336 could store information on a history of charge statuses of the battery 338. According to an embodiment, the probe 300 may send such information regarding the history of charge statuses to a computing device to which it is paired, or to a cloud network. According to an embodiment, processing circuitry 326 of probe 300 may cause a set of charge statuses stored in the memory circuitry 336 to be sent to the computing device, the set corresponding for example to charge statuses corresponding to given preset or setting of the probe 300, by way of example.

The sensor circuitry may for example include a location tracking system, such as, a Global Positioning System (GPS) device to track location (e.g., current location, and/or movement related information, such as speed and direction of movement.

For example the sensor circuitry may include a magnetometer to sense an ambient magnetic field of the earth to allow determination of location relative to an earth's pole.

For example, the sensor circuitry may be configured to detect location information of the probe, and the processing circuitry 326 may then cause the probe to send information regarding such location to the computing device.

For example, the sensor circuitry may be configured to detect location information of the probe, and the processing circuitry 326 may then cause the probe to send information regarding such location to memory circuitry 336, including information on at least one of the date, time, location duration, and/or an identifier for the location. Thus, memory circuitry 336 could store information on a history of locations of the probe 300. According to an embodiment, the probe 300 may send such information regarding the history of locations to a computing device to which it is paired, or to a cloud network. As used herein “information regarding location” may include information regarding a physical or geographic location, including indoor location, outdoor location, and, in addition, information regarding movement, such as speed and direction of movement, and information regarding travel from point A to point B.

For example, the sensor circuitry may include circuitry to detect a pairing of the ultrasonic imaging probe 300 to a computing device. The pairing may be sensed through a an exchange of signals (e.g., signals that allow the probe and the computing system to exchange their respective IDs, including for example any process, task, application or software IDs particular to a connection of the probe to the computing device) between the ultrasonic imaging probe 300 and a computing system to establish an association of the ultrasonic imaging probe 300 to the computing system.

For example, the processing circuitry of the probe may cause the probe to send information regarding such pairing to the computing device.

For example a processing circuitry of the probe may be configured to cause the probe to send information regarding the pairing for storage within a memory circuitry of the computing device, or up to a cloud-based network for storage therein. For example, each time the ultrasonic imaging probe 300 is paired with a computing device 216 (see FIG. 2), the sensor circuitry could detect such pairing, and cause the probe to send information regarding such pairing to at least one of the computing device and/or a cloud-based network, including information on at least one of the date, time, pairing duration, and/or an identifier for the computing device to which the probe 300 has been paired.

For example, each time the ultrasonic imaging probe 300 is paired with a computing device 216 (see FIG. 2), the sensor circuitry could detect such pairing, and the processing circuitry 326 may then cause the probe to send information regarding such pairing to the memory circuitry 336, including information on at least one of the date, time, pairing duration, and/or an identifier for the computing device to which the probe 300 has been paired. Thus, memory circuitry 336 could store information on a history of pairings of the probe 300 to various computing devices, such history including at least one of the date, time, pairing duration, and/or an identifier for the computing device to which the probe 300 has been paired. According to an embodiment, the probe 300 may send such information regarding the history of pairings to a computing device to which it is paired, or to a cloud network.

The I/O circuitry 334 may be adapted to send a signal (hereinafter “ultrasonic imaging probe signal”) including information to identify the ultrasonic imaging probe, and information on one or more dynamic parameters of the ultrasonic imaging probe 300, such as the dynamic parameters described above, and/or such as dynamic parameters determined by the sensor circuitry 335, to a computing device to which it is paired, such as computing device 216 (FIG. 2), and/or to a cloud network. For example, computing device 216 may be part of a cloud network, and may further send dynamic information of probe 300 to other nodes of the cloud network. The computing device 216 may include its own I/O circuitry 217 to receive the ultrasonic imaging probe signals. The computing device 216 may further include processing circuitry to generate a response signal based on the ultrasonic imaging probe signal.

According to an embodiment, the response signal may include a signal to be sent to the ultrasonic imaging probe to cause changes to an operation of the ultrasonic imaging probe (hereinafter, “operational changes to the ultrasonic imaging probe”). As used herein, “operational changes to the ultrasonic imaging probe” means changes resulting from changes in one or more settings of the ultrasonic imaging probe. Changes in the settings may result in changes in at least one of a power consumption by the probe (e.g., including power consumption by one or more components of the ultrasonic imaging probe), a temperature of the probe (e.g., including a temperature of one or more respective components of the ultrasonic imaging probe), or a dynamic battery status (e.g., level of charge in battery, whether battery being charged, frequency of battery charge within a given time period, etc.) of the ultrasonic imaging probe.

For example, the response signal may include a control signal to change an ultrasonic imaging probe setting. For example, the control signal may include a signal to cause a change in at least one of the ultrasonic imaging probe settings a) through p) mentioned above, namely at least one of: gain, depth, frequency, TGC, dynamic range, focus, harmonics, mode, focal zone, persistence, AGC, spatial compounding, frequency compounding, sine functions, line density, or tint maps.

For example, the response signal may include a control signal to change an ultrasonic imaging probe setting including at least one of: a setting of one or more transducer elements; a coupling of TX channels to one or more transducer elements, a coupling of RX channels to one or more transducer elements, a power mode of the ultrasonic imaging probe, or a communication setting of the ultrasonic imaging probe.

For example, the response signal may include a signal to the user of the probe to take an action, such as returning the probe to a given location, charging the probe's battery, changing a setting of the probe, etc.

A settings of one or more transducer elements may include at least one of:
a. Electronic focusing: One way to control individual transducer elements is through electronic focusing. This technique involves adjusting the timing and amplitude of each transducer element to create a focused beam. By controlling the timing and amplitude of each element, the beam can be focused at different depths. Presets can be used to adjust the focus of the beam for specific applications, such as imaging different organs;
b. Beam steering: Another way to control individual transducer elements is through beam steering. This technique involves adjusting the phase of each transducer element to steer the beam in a specific direction. By controlling the phase of each element, the beam can be steered to different angles. Presets can be used to adjust the direction of the beam for specific applications, such as imaging a specific area of the body;
c. Apodization: Apodization is a technique that involves adjusting the amplitude of each transducer element to improve the quality of the image. By adjusting the amplitude of each element, the unwanted side lobes of the beam can be reduced, resulting in a clearer image. Presets can be used to adjust the amplitude of each element for specific applications, such as imaging a specific organ;
d. Pulse repetition frequency (PRF): The PRF is the number of pulses sent out by the transducer per second. Presets can be used to adjust the PRF for specific applications, such as imaging a fast-moving organ like the heart; or
e. Power output: The power output of the transducer can be adjusted based on presets. This can be used to control the depth of penetration of the ultrasound waves, as higher power output will penetrate deeper into the tissue. Presets can be used to adjust the power output for specific applications, such as imaging deeper tissues or superficial tissues.

A change in a power mode of the ultrasonic imaging probe may include the ultrasonic imaging probe going between any two of an active/awake mode, a low power mode, a sleep mode, and an off mode. Each of the power modes noted may include a number of sub-modes. For example, the active/awake mode may include a mode with no power restrictions (e.g., when plugged into an electrical power source, or when battery charge percentage above a given threshold), a number of low-power or power-restricted modes (e.g. where certain settings are not accessible in order to save power or lower temperature), a sleep mode (e.g., a mode in which the probe is “on” but where functions are dormant), and an off mode (a mode in which the device is powered off).

A change in a communication setting of the ultrasonic imaging probe may include the ultrasonic imaging probe going between communication mode and a non-communication mode. In a communication mode, the ultrasonic imaging probe may be configured to communicate through the communication circuitry and the I/O circuitry with an external device, such as with a computing device. In a non-communication mode, the communication circuitry of the ultrasonic imaging probe and/or its I/O circuitry may be disabled (e.g., by way of the control signal from the computing device) such that the ultrasonic imaging probe does not send signals to the external device. It may, for example, store imaging signals inside its memory circuitry (e.g., memory circuitry 336) and communicate them only upon the satisfaction of certain dynamic conditions, such as when the battery charge is above a certain percentage, or such as when the ultrasonic imaging probe is being powered through an external power source (such as by being plugged into a power socket).

For example, when the ultrasonic imaging probe signal indicates a dynamic parameter including information that the battery of the ultrasonic imaging probe has charge percentage of less than a predetermined number, such as, say 10% or 15%, a response signal back to the ultrasonic imaging probe may include a control signal with a command to disable a radio (e.g., an example of the communication circuitry 332) of the ultrasonic imaging probe until the battery charge percentage increases to the predetermined threshold. Concurrently, the control signal may include a command to the processing circuitry to store any imaging signals by the ultrasonic imaging probe during the time that the radio is disabled to the memory circuitry 336.

For example, when the ultrasonic imaging probe signal indicates a dynamic parameter indicating location information regarding the ultrasonic imaging probe indicating that the ultrasonic imaging probe is beyond a predetermined location (e.g., a predetermined geofenced area, a hospital, a battlefield area, a rescue operation area), the response signal from the computing device may include a control signal to disable the ultrasonic imaging probe, or to cause the ultrasonic imaging probe to communicate (e.g., via vibration, a signature noise, or otherwise) to a user that it is to be returned to its predetermined location.

According to an embodiment, the ultrasonic imaging probe may be configured to send an ultrasonic imaging probe signal with information regarding the one or more dynamic parameters at predetermined regular intervals, at intervals as configured by the computing device to which it is paired, and/or based on a query or request by the computing device. For example, the computing device may, after a predetermined amount of time has elapsed, query the ultrasonic probe for one or more of its dynamic parameters.

According to an embodiment, the response signal may include a signal to be sent to one or more nodes of a cloud network for storage and/or processing at the cloud network. The signal to be sent to one or more nodes of a cloud network may correspond to a relaying of the information within the ultrasonic imaging probe signal that includes the information one the one or more dynamic parameters, and/or it may include signals based on a processing, by a processing circuitry of the computing device, of the information within the ultrasonic imaging probe signals.

Some embodiments advantageously allow a monitoring of an ultrasonic imaging probe based on one or more dynamic parameters thereof and a control of the probe in order to bring operational changes thereto based on the one or more dynamic parameters, in this manner improving a technical performance of the probe while extending its operational life.

FIG. 4 shows a method 400 to be performed at an ultrasound imaging device according to one embodiment. The method includes, at operation 402, sending, for transmission from the probe to a computing device, an ultrasonic imaging probe signal including an identification (ID) of the probe, and one or more dynamic parameters of the probe; at operation 404, sending, for transmission from the probe to a computing device, an ultrasonic imaging probe signal including an identification (ID) of the probe, and one or more dynamic parameters of the probe; and at operation 406, causing an operational change at the probe based on the control signal, the operational change to cause at least one of a change in a power consumption by the probe, a change in a temperature at the probe, or a change in a battery status at the probe.

FIG. 5 shows a method 500 to be performed at computing device according to one embodiment. The method includes, at operation 502, receiving, from the ultrasonic imaging probe, an ultrasonic imaging probe signal including an identification (ID) of the probe, and one or more dynamic parameters of the probe; at operation 504, sending for transmission, from the computing device, a control signal based on the ultrasonic imaging probe signal; and at operation 506, encoding the control signal for transmission to the probe, the control signal, when decoded at the probe, to cause an operational change at the probe resulting in at least one of a change in a power consumption by the probe, a change in a temperature at the probe, or a change in a battery status at the probe.

The flows described in FIG. 4 and FIG. 5 are merely representative of operations that may occur in particular embodiments. In other embodiments, additional operations may be performed by the components of the systems shown in FIGS. 1-3. Various embodiments of the present disclosure contemplate any suitable mechanisms for accomplishing the functions described herein. Some of the operations illustrated in FIG. 4 may be repeated, combined, modified, or deleted where appropriate. Additionally, operations may be performed in any suitable order without departing from the scope of particular embodiments.

A design may go through various stages, from creation to simulation to fabrication. Data representing a design may represent the design in a number of manners. First, as is useful in simulations, the hardware may be represented using a hardware description language (HDL) or another functional description language. Additionally, a circuit level model with logic and/or transistor gates may be produced at some stages of the design process. Furthermore, most designs, at some stage, reach a level of data representing the physical placement of various devices in the hardware model. In some implementations, such data may be stored in a database file format such as Graphic Data System II (GDS II), Open Artwork System Interchange Standard (OASIS), or similar format.

In any representation of the design, the data may be stored in any form of a machine readable medium. A memory or a magnetic or optical storage such as a disc may be the machine readable medium to store information transmitted via optical or electrical wave modulated or otherwise generated to transmit such information. When an electrical carrier wave indicating or carrying the code or design is transmitted, to the extent that copying, buffering, or re-transmission of the electrical signal is performed, a new copy is made. Thus, a communication provider or a network provider may store on a tangible, machine-readable medium, at least temporarily, an article, such as information encoded into a carrier wave, embodying techniques of embodiments of the present disclosure.

In various embodiments, a medium storing a representation of the design may be provided to a manufacturing system (e.g., a semiconductor manufacturing system capable of manufacturing an integrated circuit and/or related components). The design representation may instruct the system to manufacture a device capable of performing any combination of the functions described above. For example, the design representation may instruct the system regarding which components to manufacture, how the components should be coupled together, where the components should be placed on the device, and/or regarding other suitable specifications regarding the device to be manufactured.

“Circuitry” as used herein may refer to any combination of hardware with software, and/or firmware. As an example, a circuitry includes hardware, such as a micro-controller, associated with a non-transitory medium to store code adapted to be executed by the micro-controller. Therefore, reference to a circuitry, in one embodiment, refers to the hardware, which is specifically configured to recognize and/or execute the code to be held on a non-transitory medium. Furthermore, in another embodiment, use of a circuitry refers to the non-transitory medium including the code, which is specifically adapted to be executed by the microcontroller to perform predetermined operations. And as can be inferred, in yet another embodiment, the term circuitry (in this example) may refer to the combination of the microcontroller and the non-transitory medium. Often circuitry boundaries that are illustrated as separate commonly vary and potentially overlap. For example, a first and a second circuitry may share hardware, software, firmware, or a combination thereof, while potentially retaining some independent hardware, software, or firmware. In one embodiment, use of the term logic includes hardware, such as transistors, registers, or other hardware, such as programmable logic devices.

Logic may be used to implement any of the flows described or functionality of the various components described herein. “Logic” may refer to hardware, firmware, software and/or combinations of each to perform one or more functions. In various embodiments, logic may include a microprocessor or other processing element operable to execute software instructions, discrete logic such as an application-specific integrated circuit (ASIC), a programmed logic device such as a field programmable gate array (FPGA), a storage device containing instructions, combinations of logic devices (e.g., as would be found on a printed circuit board), or other suitable hardware and/or software. Logic may include one or more gates or other circuit components. In some embodiments, logic may also be fully embodied as software. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in storage devices.

Use of the phrase ‘to’ or ‘configured to,’ in one embodiment, refers to arranging, putting together, manufacturing, offering to sell, importing, and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task. As a purely illustrative example, a logic gate may provide a 0 or a 1 during operation. But a logic gate ‘configured to’ provide an enable signal to a clock does not include every potential logic gate that may provide a 1 or 0. Instead, the logic gate is one coupled in some manner that during operation the 1 or 0 output is to enable the clock. Note once again that use of the term ‘configured to’ does not require operation, but instead focuses on the latent state of an apparatus, hardware, and/or element, wherein the latent state the apparatus, hardware, and/or element is designed to perform a particular task when the apparatus, hardware, and/or element is operating.

Furthermore, use of the phrases ‘capable of/to,’ and or ‘operable to,’ in one embodiment, refers to some apparatus, logic, hardware, and/or element designed in such a way to enable use of the apparatus, logic, hardware, and/or element in a specified manner. Note as above that use of to, capable to, or operable to, in one embodiment, refers to the latent state of an apparatus, logic, hardware, and/or element, where the apparatus, logic, hardware, and/or element is not operating but is designed in such a manner to enable use of an apparatus in a specified manner.

The embodiments of methods, hardware, software, firmware, or code set forth above may be implemented via instructions or code stored on a machine-accessible, machine readable, computer accessible, or computer readable medium which are executable by a processing element. A tangible non-transitory machine-accessible/readable medium includes any mechanism that provides (i.e., stores and/or transmits) information in a form readable by a machine, such as a computer or electronic system. For example, a non-transitory machine-accessible medium includes random-access memory (RAM), such as static RAM (SRAM) or dynamic RAM (DRAM); ROM; magnetic or optical storage medium; flash storage devices; electrical storage devices; optical storage devices; acoustical storage devices; other form of storage devices for holding information received from transitory (propagated) signals (e.g., carrier waves, infrared signals, digital signals); etc., which are to be distinguished from the non-transitory mediums that may receive information therefrom.

Instructions used to program logic to perform embodiments of the disclosure may be stored within a memory in the system, such as DRAM, cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, Compact Disc, Read-Only Memory (CD-ROMs), and magneto-optical disks, Read-Only Memory (ROMs), Random Access Memory (RAM), Erasable Programmable Read-Only Memory (EPROM), Electrically Erasable Programmable Read-Only Memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the computer-readable medium includes any type of tangible machine-readable medium suitable for storing or transmitting electronic instructions or information in a form readable by a machine (e.g., a computer).

Some embodiments include means for performing any of the operations described herein.

Some example embodiments will now be described below.

### EXAMPLES

Illustrative examples of the technologies disclosed herein are provided below. An embodiment of the technologies may include any one or more, and any combination of, the examples described below.

Example 1 includes an ultrasonic imaging probe including: a plurality of ultrasonic transducer elements to generate and transmit ultrasonic waveforms in a direction of a target to be imaged; control circuitry to control the transducer elements; communication circuitry to: send, for transmission from the probe to a computing device, an ultrasonic imaging probe signal including an identification (ID) of the probe, and one or more dynamic parameters of the probe; receive from the computing device a control signal based on the ultrasonic imaging probe signal; and processing circuitry to cause an operational change at the probe based on the control signal, the operational change to cause at least one of a change in a power consumption by the probe, a change in a temperature at the probe, or a change in a battery status at the probe.

Example 2 includes the subject matter of Example 1, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

Example 3 includes the subject matter of any one of Examples 1-2, wherein an operational change at the probe corresponds to a change in at least one of: a setting of one or more of the transducer elements; a power mode of the probe, or a communication setting of the probe.

Example 4 includes the subject matter of Example 2, further including a battery to supply power to the probe.

Example 5 includes the subject matter of Example 4, wherein the probe includes a sensor circuitry to sense the one or more dynamic parameters, and the one or more dynamic parameters include one or more respective temperatures at one or more corresponding ones of the transducer elements, the control circuitry, the communication circuitry, the processing circuitry, the sensor circuitry or the battery.

Example 6 includes the subject matter of any one of Examples 4-5, wherein the probe includes a sensor circuitry to sense the one or more dynamic parameters, and the one or more dynamic parameters include one or more respective voltages at one or more corresponding ones of the transducer elements, the control circuitry, the communication circuitry, the processing circuitry, the sensor circuitry or the battery.

Example 7 includes the subject matter of any one of Examples 1-6, wherein the one or more dynamic parameters include at least one of a battery charge status of the probe, a location of the probe, or pairing information regarding a pairing status of the probe to the computing device.

Example 8 includes the subject matter of Example 7, further including memory circuitry to store a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter, the processing circuitry to access the history, the ultrasonic imaging probe signal including the history of the dynamic parameter.

Example 9 includes the subject matter of Example 8, wherein, in response to a determination that the one or more dynamic parameters indicate the battery charge status having a charge percentage less than or equal to a predetermined value, the control signal includes a command to disable the communication circuitry, the processing circuitry to enable the communication circuitry in response to a determination that a battery charge percentage of the probe is equal to or more than a predetermined threshold.

Example 10 includes the subject matter of Example 9, wherein the control signal further includes a command to the processing circuitry to store, in the memory circuitry and during a time that the communication circuitry is disabled, imaging signals by the ultrasonic imaging probe.

Example 11 includes the subject matter of any one of Examples 7-10, wherein, in response to a determination that the one or more dynamic parameters indicate that a location of the probe is beyond a predetermined area, the control signal includes a command to one of disable the ultrasonic imaging probe.

Example 12 includes the subject matter of any one of Examples 1-11, wherein the communication circuitry is to send the ultrasonic imaging probe signal at least one of based on a predetermined cadence or in response to a query by the computing device.

Example 13 includes the subject matter of any one of Examples 1-12, further including sensor circuitry to sense the one or more dynamic parameters.

Example 14 includes a method to be performed at an ultrasonic imaging probe including: sending, for transmission from the probe to a computing device, an ultrasonic imaging probe signal including an identification (ID) of the probe, and one or more dynamic parameters of the probe; receiving from the computing device a control signal based on the ultrasonic imaging probe signal; and causing an operational change at the probe based on the control signal, the operational change to cause at least one of a change in a power consumption by the probe, a change in a temperature at the probe, or a change in a battery status at the probe.

Example 15 includes the subject matter of Example 14, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

Example 16 includes the subject matter of any one of Examples 14-15, wherein an operational change at the probe corresponds to a change in at least one of: a setting of one or more of transducer elements of the probe; a power mode of the probe, or a communication setting of the probe.

Example 17 includes the subject matter of Example 16, wherein the one or more dynamic parameters include one or more respective temperatures at one or more of the transducer elements, at a control circuitry of the probe to control the transducer elements, at a communication circuitry of the probe to communicate with the computing device, at a processing circuitry that is to cause the operational change, at a sensor circuitry of the probe to sense the one or more dynamic parameters, or a battery of the probe.

Example 18 includes the subject matter of Example 17, wherein the probe includes a sensor circuitry to sense the one or more dynamic parameters, and the one or more dynamic parameters include one or more respective voltages at one or more corresponding ones of the transducer elements, the control circuitry, the communication circuitry, the processing circuitry, the sensor circuitry or the battery.

Example 19 includes the subject matter of any one of Examples 14-18, wherein the one or more dynamic parameters include at least one of a battery charge status of the probe, a location of the probe, or pairing information regarding a pairing status of the probe to the computing device.

Example 20 includes the subject matter of Example 19, further including: storing, at a memory circuitry of the probe, a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter; and accessing the history, the ultrasonic imaging probe signal including the history of the dynamic parameter.

Example 21 includes the subject matter of Example 20, wherein, in response to a determination that the one or more dynamic parameters indicate the battery charge status having a charge percentage less than or equal to a predetermined value, disabling, based on the control signal, communication to and from the probe based, and enabling communication to and from the probe in response to a determination that a battery charge percentage of the probe is equal to or more than a predetermined threshold.

Example 22 includes the subject matter of Example 21, further including storing, based on the control signal, in the memory circuitry and during a time that communication to and from the probe is disabled, imaging signals by the ultrasonic imaging probe.

Example 23 includes the subject matter of any one of Examples 19-22, further including, in response to a determination that the one or more dynamic parameters indicate that a location of the probe is beyond a predetermined area, disabling, based on the control signal, the ultrasonic imaging probe.

Example 24 includes the subject matter of any one of Examples 14-23, wherein the communication circuitry is to send the ultrasonic imaging probe signal at least one of based on a predetermined cadence or in response to a query by the computing device.

Example 25 includes the subject matter of any one of Examples 14-24, further including sensing the one or more dynamic parameters.

Example 26 includes a product comprising one or more tangible computer-readable non-transitory storage media comprising computer-executable instructions operable to, when executed by at least one processing circuitry of an ultrasonic imaging probe, cause the at least one processing circuitry to implement operations at the ultrasonic imaging probe, the operations comprising: encoding, for transmission from the probe to a computing device distinct from the probe, an ultrasonic imaging probe signal including an identification (ID) of the probe, and one or more dynamic parameters of the probe; decoding a control signal from the computing device, the control signal based on the ultrasonic imaging probe signal; and causing an operational change at the probe based on the control signal, the operational change to cause at least one of a change in a power consumption by the probe, a change in a temperature at the probe, or a change in a battery status at the probe.

Example 27 includes the subject matter of Example 26, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

Example 28 includes the subject matter of any one of Examples 26-27, wherein an operational change at the probe corresponds to a change in at least one of: a setting of one or more of transducer elements of the probe; a power mode of the probe, or a communication setting of the probe.

Example 29 includes the subject matter of Example 28, wherein the probe includes a sensor circuitry to sense the one or more dynamic parameters, and the one or more dynamic parameters include one or more respective temperatures at one or more of the transducer elements, a control circuitry of the probe to control the transducer elements, a communication circuitry of the probe to communicate with the computing device, a processing circuitry to cause the operational change, a sensor circuitry to sense the one or more dynamic parameters, or a battery of the probe.

Example 30 includes the subject matter of Example 29, wherein the probe includes a sensor circuitry to sense the one or more dynamic parameters, and the one or more dynamic parameters include one or more respective voltages at one or more corresponding ones of the transducer elements, the control circuitry, the communication circuitry, the processing circuitry, the sensor circuitry or the battery.

Example 31 includes the subject matter of any one of Examples 26-30, wherein the one or more dynamic parameters include at least one of a battery charge status of the probe, a location of the probe, or pairing information regarding a pairing status of the probe to the computing device.

Example 32 includes the subject matter of Example 31, further including: causing to store, at a memory circuitry of the probe, a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter; and accessing the history, the ultrasonic imaging probe signal including the history of the dynamic parameter.

Example 33 includes the subject matter of Example 32, wherein, in response to a determination that the one or more dynamic parameters indicate the battery charge status having a charge percentage less than or equal to a predetermined value, in response to a determination that the one or more dynamic parameters indicate the battery charge status having a charge percentage less than or equal to a predetermined value, disabling, based on the control signal, communication to and from the probe based, and enabling communication to and from the probe in response to a determination that a battery charge percentage of the probe is equal to or more than a predetermined threshold.

Example 34 includes the subject matter of Example 33, further including causing to store, based on the control signal, in the memory circuitry and during a time that communication to and from the probe is disabled, imaging signals by the ultrasonic imaging probe.

Example 35 includes the subject matter of any one of Examples 31-34, further including, in response to a determination that the one or more dynamic parameters indicate that a location of the probe is beyond a predetermined area, disabling, based on the control signal, the ultrasonic imaging probe.

Example 36 includes the subject matter of any one of Examples 26-35, further including decoding the ultrasonic imaging probe signal at least one of based on a predetermined cadence of receipt of the ultrasonic imaging probe signal at the probe, or prior to decoding the ultrasonic imaging probe signal, encoding, for transmission to the computing device, a query for the ultrasonic imaging probe signal.

Example 37 includes the subject matter of any one of Examples 26-36, further including sensing the one or more dynamic parameters.

Example 38 includes a computing device to be in communication with an ultrasonic imaging probe, the computing device including: communication circuitry to: receive, from the ultrasonic imaging probe, an ultrasonic imaging probe signal including an identification (ID) of the probe, and one or more dynamic parameters of the probe; send for transmission, from the computing device, a control signal based on the ultrasonic imaging probe signal; and processing circuitry to encode the control signal for transmission to the probe, the control signal, when decoded at the probe, to cause an operational change at the probe resulting in at least one of a change in a power consumption by the probe, a change in a temperature at the probe, or a change in a battery status at the probe.

Example 39 includes the subject matter of Example 38, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

Example 40 includes the subject matter of any one of Examples 38-39, wherein an operational change at the probe corresponds to a change in at least one of: a setting of one or more of transducer elements of the probe; a power mode of the probe, or a communication setting of the probe.

Example 41 includes the subject matter of any one of Examples 38-40, wherein the probe includes a sensor circuitry to sense the one or more dynamic parameters, and the one or more dynamic parameters include one or more respective temperatures at one or more corresponding ones of transducer elements of the probe, a control circuitry of the probe to control the transducer elements, a communication circuitry of the probe, a processing circuitry of the probe, a sensor circuitry of the probe, or a battery of the probe.

Example 42 includes the subject matter of Example 41, wherein the probe includes a sensor circuitry to sense the one or more dynamic parameters, and the one or more dynamic parameters include one or more respective voltages at one or more corresponding ones of the transducer elements of the probe, the control circuitry of the probe, the communication circuitry of the probe, the processing circuitry of the probe, the sensor circuitry or the battery of the probe.

Example 43 includes the subject matter of any one of Examples 38-42, wherein the one or more dynamic parameters include at least one of a battery charge status of the probe, a location of the probe, or pairing information regarding a pairing status of the probe to the computing device.

Example 44 includes the subject matter of Example 43, further including memory circuitry to store a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter, the processing circuitry of the computing device to send the history to another node in a cloud network.

Example 45 includes the subject matter of Example 44, wherein, in response to a determination that the one or more dynamic parameters indicate the battery charge status having a charge percentage less than or equal to a predetermined value, the control signal includes a command to disable the communication circuitry of the probe.

Example 46 includes the subject matter of any one of Examples 44-45, wherein, in response to a determination that the one or more dynamic parameters indicate that a location of the probe is beyond a predetermined area, the control signal includes a command to disable the ultrasonic imaging probe.

Example 47 includes the subject matter of any one of Examples 38-46, wherein the communication circuitry of the computing device is to one of: expect to receive the ultrasonic imaging probe signal at least one of based on a predetermined cadence; or encode for transmission to the probe a query to cause the probe to send the ultrasonic imaging probe signal based on the query.

Example 48 includes a method to be performed at a computing device in communication with an ultrasonic imaging probe, the method including: receiving, from the ultrasonic imaging probe, an ultrasonic imaging probe signal including an identification (ID) of the probe, and one or more dynamic parameters of the probe; sending for transmission, from the computing device, a control signal based on the ultrasonic imaging probe signal; and encoding the control signal for transmission to the probe, the control signal, when decoded at the probe, to cause an operational change at the probe resulting in at least one of a change in a power consumption by the probe, a change in a temperature at the probe, or a change in a battery status at the probe.

Example 49 includes the subject matter of Example 48, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

Example 50 includes the subject matter of any one of Examples 48-49, wherein an operational change at the probe corresponds to a change in at least one of: a setting of one or more transducer elements of the probe; a power mode of the probe, or a communication setting of the probe.

Example 51 includes the subject matter of any one of Examples 48-50, wherein the probe includes a sensor circuitry to sense the one or more dynamic parameters, and the one or more dynamic parameters include one or more respective temperatures at one or more corresponding ones of transducer elements of the probe, a control circuitry of the probe to control the transducer elements, a communication circuitry of the probe, a processing circuitry of the probe, a sensor circuitry of the probe, or a battery of the probe.

Example 52 includes the subject matter of Example 51, wherein the probe includes a sensor circuitry to sense the one or more dynamic parameters, and the one or more dynamic parameters include one or more respective voltages at one or more corresponding ones of the transducer elements of the probe, the control circuitry of the probe, the communication circuitry of the probe, the processing circuitry of the probe, the sensor circuitry or the battery of the probe.

Example 53 includes the subject matter of any one of Examples 48-52, wherein the one or more dynamic parameters include at least one of a battery charge status of the probe, a location of the probe, or pairing information regarding a pairing status of the probe to the computing device.

Example 54 includes the subject matter of Example 53, further including: storing in a memory circuitry a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter; and sending the history to another node in a cloud network.

Example 55 includes the subject matter of Example 54, wherein, in response to a determination that the one or more dynamic parameters indicate the battery charge status having a charge percentage less than or equal to a predetermined value, the control signal includes a command to disable communication to or from the probe.

Example 56 includes the subject matter of any one of Examples 54-55, wherein, in response to a determination that the one or more dynamic parameters indicate that a location of the probe is beyond a predetermined area, the control signal includes a command to disable the ultrasonic imaging probe.

Example 57 includes the subject matter of any one of Examples 48-56, further including one of: expecting to receive the ultrasonic imaging probe signal at least one of based on a predetermined cadence; or encoding for transmission to the probe a query to cause the probe to send the ultrasonic imaging probe signal based on the query.

Example 58 includes a product comprising one or more tangible computer-readable non-transitory storage media comprising computer-executable instructions operable to, when executed by at least one processing circuitry of a computing device to be communicatively paired with an ultrasonic imaging probe, cause the at least one processing circuitry to implement operations at the computing device, the operations comprising: receiving, from the ultrasonic imaging probe, an ultrasonic imaging probe signal including an identification (ID) of the probe, and one or more dynamic parameters of the probe; sending for transmission, from the computing device, a control signal based on the ultrasonic imaging probe signal; and encoding the control signal for transmission to the probe, the control signal, when decoded at the probe, to cause an operational change at the probe resulting in at least one of a change in a power consumption by the probe, a change in a temperature at the probe, or a change in a battery status at the probe.

Example 59 includes the subject matter of Example 58, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

Example 60 includes the subject matter of any one of Examples 58-59, wherein an operational change at the probe corresponds to a change in at least one of: a setting of one or more transducer elements of the probe; a power mode of the probe, or a communication setting of the probe.

Example 61 includes the subject matter of any one of Examples 58-60, wherein the probe includes a sensor circuitry to sense the one or more dynamic parameters, and the one or more dynamic parameters include one or more respective temperatures at one or more corresponding ones of transducer elements of the probe, a control circuitry of the probe to control the transducer elements, a communication circuitry of the probe, a processing circuitry of the probe, a sensor circuitry of the probe, or a battery of the probe.

Example 62 includes the subject matter of Example 61, wherein the probe includes a sensor circuitry to sense the one or more dynamic parameters, and the one or more dynamic parameters include one or more respective voltages at one or more corresponding ones of the transducer elements of the probe, the control circuitry of the probe, the communication circuitry of the probe, the processing circuitry of the probe, the sensor circuitry or the battery of the probe.

Example 63 includes the subject matter of any one of Examples 58-62, wherein the one or more dynamic parameters include at least one of a battery charge status of the probe, a location of the probe, or pairing information regarding a pairing status of the probe to the computing device.

Example 64 includes the subject matter of Example 63, the operations further including storing in a memory circuitry a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter, the processing circuitry of the computing device to send the history to another node in a cloud network.

Example 65 includes the subject matter of Example 64, wherein, in response to a determination that the one or more dynamic parameters indicate the battery charge status having a charge percentage less than or equal to a predetermined value, the control signal includes a command to disable communication to or from the probe.

Example 66 includes the subject matter of any one of Examples 64-65, wherein, in response to a determination that the one or more dynamic parameters indicate that a location of the probe is beyond a predetermined area, the control signal includes a command to disable the ultrasonic imaging probe.

Example 67 includes the subject matter of any one of Examples 58-66, the operations further including one of: expecting to receive the ultrasonic imaging probe signal at least one of based on a predetermined cadence; or encoding for transmission to the probe a query to cause the probe to send the ultrasonic imaging probe signal based on the query.

Example 68 includes one or more computer-readable media comprising instructions stored thereon that, when executed, cause one or more processors to perform the method of any one of Examples 14-25 or 48-57.

Example 69 includes an imaging device comprising the apparatus of any one of Examples 14-25 or 48-57, and further including the user interface device.

Example 70 includes a product comprising one or more tangible computer-readable non-transitory storage media comprising computer-executable instructions operable to, when executed by at least one computer processor, enable the at least one processor to perform the method of any one of Examples 14-25 or 48-57.

Example 71 includes an apparatus comprising means for performing the method of any one of the method Examples above.

What is claimed is:

1. An ultrasonic imaging probe including:
a plurality of ultrasonic transducer elements to generate and transmit ultrasonic waveforms in a direction of a target to be imaged;
control circuitry to control the transducer elements;
communication circuitry to:
send, for transmission from the probe to a computing device, an ultrasonic imaging probe signal including an identification (ID) of the probe, and one or more dynamic parameters of the probe;
receive from the computing device a control signal based on the ultrasonic imaging probe signal; and
processing circuitry to cause an operational change at the probe based on the control signal, the operational change to cause at least one of a change in a power consumption by the probe, a change in a temperature at the probe, or a change in a battery status at the probe.

2. The ultrasonic imaging probe of claim 1, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

3. The ultrasonic imaging probe of claim 2, wherein an operational change at the probe corresponds to a change in at least one of: a setting of one or more of the transducer elements; a power mode of the probe, or a communication setting of the probe.

4. The ultrasonic imaging probe of claim 3, further including a battery to supply power to the probe.

5. The ultrasonic imaging probe of claim 4, wherein the probe includes a sensor circuitry to sense the one or more dynamic parameters, and the one or more dynamic parameters include one or more respective temperatures at one or more corresponding ones of the transducer elements, the control circuitry, the communication circuitry, the processing circuitry, the sensor circuitry or the battery.

6. The ultrasonic imaging probe of claim 4, wherein the probe includes a sensor circuitry to sense the one or more dynamic parameters, and the one or more dynamic parameters include one or more respective voltages at one or more corresponding ones of the transducer elements, the control circuitry, the communication circuitry, the processing circuitry, the sensor circuitry or the battery.

7. The ultrasonic imaging probe of claim 1, wherein the one or more dynamic parameters include at least one of a battery charge status of the probe, a location of the probe, or pairing information regarding a pairing status of the probe to the computing device.

8. The ultrasonic imaging probe of claim 7, further including memory circuitry to store a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter, the processing circuitry to access the history, the ultrasonic imaging probe signal including the history of the dynamic parameter.

9. The ultrasonic imaging probe of claim 8, wherein, in response to a determination that the one or more dynamic parameters indicate the battery charge status having a charge percentage less than or equal to a predetermined value, the control signal includes a command to disable the communication circuitry, the processing circuitry to enable the communication circuitry in response to a determination that a battery charge percentage of the probe is equal to or more than a predetermined threshold.

10. The ultrasonic imaging probe of claim 9, wherein the control signal further includes a command to the processing circuitry to store, in the memory circuitry and during a time that the communication circuitry is disabled, imaging signals by the ultrasonic imaging probe.

11. The ultrasonic imaging probe of claim 7, wherein, in response to a determination that the one or more dynamic parameters indicate that a location of the probe is beyond a predetermined area, the control signal includes a command to one of disable the ultrasonic imaging probe.

12. The ultrasonic imaging probe of claim 1, wherein the communication circuitry is to send the ultrasonic imaging probe signal at least one of based on a predetermined cadence or in response to a query by the computing device.

13. The ultrasonic imaging probe of claim 1, further including sensor circuitry to sense the one or more dynamic parameters.

14. A method to be performed at an ultrasonic imaging probe including:
sending, for transmission from the probe to a computing device, an ultrasonic imaging probe signal including an identification (ID) of the probe, and one or more dynamic parameters of the probe;
receiving from the computing device a control signal based on the ultrasonic imaging probe signal; and
causing an operational change at the probe based on the control signal, the operational change to cause at least one of a change in a power consumption by the probe, a change in a temperature at the probe, or a change in a battery status at the probe.

15. The method of claim 14, wherein an operational change at the probe corresponds to a change in at least one setting of the probe, a setting of the probe including gain, depth, frequency, time gain compensation, dynamic range, focus, harmonics, mode, focal zone, persistence, automatic gain control, spatial compounding, frequency compounding, sine functions, line density, or tint map configuration.

16. The method of claim 14, wherein an operational change at the probe corresponds to a change in at least one of: a setting of one or more of transducer elements of the probe; a power mode of the probe, or a communication setting of the probe.

17. The method of claim 16, wherein the one or more dynamic parameters include one or more respective temperatures at one or more of the transducer elements, at a control circuitry of the probe to control the transducer elements, at a communication circuitry of the probe to communicate with the computing device, at a processing circuitry that is to cause the operational change, at a sensor circuitry of the probe to sense the one or more dynamic parameters, or a battery of the probe.

18. The method of claim 17, wherein the probe includes a sensor circuitry to sense the one or more dynamic parameters, and the one or more dynamic parameters include one or more respective voltages at one or more corresponding ones of the transducer elements, the control circuitry, the communication circuitry, the processing circuitry, the sensor circuitry or the battery.

19. The method of claim 14, wherein the one or more dynamic parameters include at least one of a battery charge status of the probe, a location of the probe, or pairing information regarding a pairing status of the probe to the computing device.

20. The method of claim 19, further including:
storing, at a memory circuitry of the probe, a dynamic parameter of the one or more dynamic parameters along with at least one of a date at which the dynamic parameter of the one or more dynamic parameters was determined, a time at which the dynamic parameter of the one or more dynamic parameters was determined, or a duration of the dynamic parameter of the one or more dynamic parameters, the memory circuitry to store a history of changes to the dynamic parameter; and
accessing the history, the ultrasonic imaging probe signal including the history of the dynamic parameter.

21. The method of claim 20, wherein, in response to a determination that the one or more dynamic parameters indicate the battery charge status having a charge percentage less than or equal to a predetermined value, disabling, based on the control signal, communication to and from the probe based, and enabling communication to and from the probe in response to a determination that a battery charge percentage of the probe is equal to or more than a predetermined threshold.

22. The method of claim 21, further including storing, based on the control signal, in the memory circuitry and during a time that communication to and from the probe is disabled, imaging signals by the ultrasonic imaging probe.

23. The method of claim 19, further including, in response to a determination that the one or more dynamic parameters indicate that a location of the probe is beyond a predetermined area, disabling, based on the control signal, the ultrasonic imaging probe.

24. One or more computer-readable media comprising a plurality of instructions stored thereon that, when executed, cause one or more processors to perform the method of claim 14.

25. An apparatus comprising means for performing the method of claim 14.
